(19) United States
(12) Patent Application Publication
(10) Pub. No.: US 2003/0045824 A1
(43) Pub. Date: Mar. 6, 2003

(54) Portable inflatable lumbar support

(76) Inventor: John Murphy, Weymouth, MA (US)

Correspondence Address: STEVEN N. FOX, P.O. Box 251, Canton, MA, 02021, US

(21) Appl. No.: 09/948,075

(22) Filed: Sep. 6, 2001

(51) Int. Cl. A61F 5/00

(52) U.S. Cl. 602/32

(57) ABSTRACT

The present invention is a portable lumbar support kit for use by a person to support the lumbar region of the person's back. The portable lumbar support kit comprises a pouch having an inner compartment and a clip member that allows the pouch to be removably attached to the belt of the person. The kit further comprises an inflatable cushion folded and stored within the pouch. When needed, the inflatable cushion may be removed from the pouch and unfolded and inflated by the person to support the lumbar region of the person's back.

### FIELD OF THE INVENTION

The present invention relates to cushions for supporting the lumbar region of the back.

### BACKGROUND OF THE INVENTION

A variety of cushions have been developed that provide support for the lumbar region of a person's back. Such cushions are not easily transportable and operational for use by a person.

### SUMMARY OF THE INVENTION

One object of the present invention was to develop a lumbar support cushion that could be easily carried by a person and used by the person as needed to provide support to the lumbar region of the person's back.

The present invention is a portable lumbar support kit for use by a person to support the lumbar region of the person's back. The portable lumbar support kit comprises a pouch having an inner compartment and a clip member that allows the pouch to be removably attached to the belt of a person. The kit further comprises an inflatable cushion folded and stored within the pouch. When needed, the inflatable cushion may be removed from the pouch and unfolded and inflated by the person to support the lumbar region of the person's back.

### BRIEF DESCRIPTION OF THE DRAWINGS

The following detailed description of the invention will be more fully understood with reference to the accompanying drawings in which:

FIG. 1 is a perspective view of the inflatable cushion;

FIG. 2 is a front elevation view of the pouch; and

FIG. 3 is a side elevation view of the pouch.

### DETAILED DESCRIPTION OF THE INVENTION

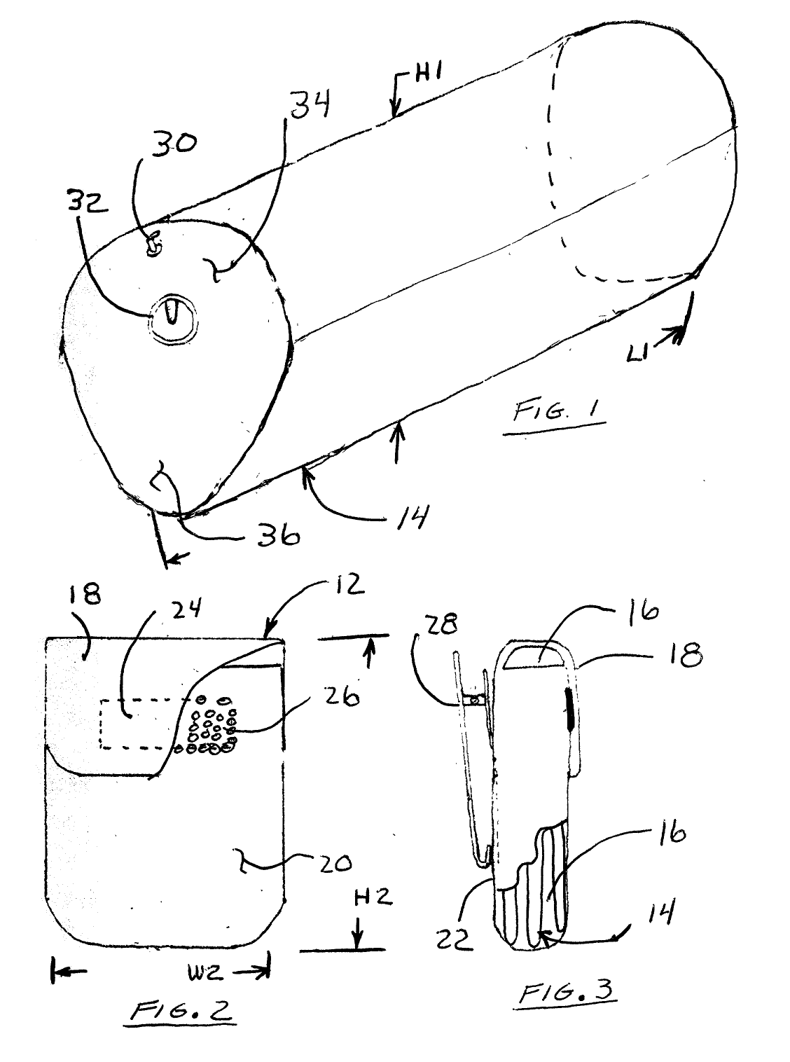

Referring to FIGS. 1 and 2, where a portable lumbar support kit 10 of the present invention is shown for carrying and use by a person as needed to support the lumbar region of the person's back. The portable lumbar support kit 10 generally comprises a pouch 12 and an inflatable cushion 14 stored therein. The pouch 12 comprises an inner compartment 16 and a cover 18. The inner compartment 16 comprises a front wall 20 and a rear wall 22. The cover 18 is engageable with the inner compartment 16 from an open position where the inflatable cushion 14 can be removed and a closed position where the inflatable cushion 14 is retained within the inner compartment 16. The cover 18 extends from the rear wall 22 of the inner compartment 16 and is removably engaged with the front wall 20 of the inner compartment 16 by hook and loop fasteners 24 and 26 so that the pouch 12 can be easily opened and closed. The pouch 12 further comprises a clip 28 mounted to the rear wall 22 of the inner compartment 16 and which is engageable with the belt of a person. The clip 28 allows the person to easily and quickly attach and detach the pouch 12 from the person's belt. The pouch 12 is made from nylon material and formed by stitching processes. It is critical to the operation of the kit 10 that the pouch 12 have a height H2 of less than three (3) inches and a width W2 of less than three (3) inches so that the pouch 12 can be comfortably carried on the person's belt.

The inflatable cushion 14 is folded and stored within the pouch 12. The inflatable cushion 14 may be unfolded and inflated by the person to support the lumbar region of the person's back. The inflatable cushion 14 comprises first and second air valves 30 and 32 adapted to inflate and deflate the inflatable cushion 14. The first air valve 30 is adapted to allow the person to inflate the inflatable cushion 16. The second valve 32 is adapted to allow the person to deflate the inflatable cushion 14. The first and second valves 30 and 32 each have an opening in communication with the inside of the inflatable cushion 12 and a stem that may be inserted therein to close the valve and removed to open the valve. The first valve 30 may be inflated by the person blowing air thru the opening and into the inflatable cushion 14. The second valve 32 is designed with a significantly larger opening than the first valve 30 so that upon opening of the second valve 32 the inflatable cushion 14 can be deflated quickly. The inflatable cushion 14 may be folded when deflated and stored within the inner compartment 16 of the pouch 12. The inflatable cushion 16 is in the form of an elongated cylinder having an upper portion 34 and a lower portion 36. The width of the lower portion is less than the width of the upper portion to provide an elongated and tapered shape. When inflated, the cushion 14 has a height H1 in the range of six (8) to eight (8) inches and a length L1 in the range of sixteen (16) to eighteen (18) inches. Dimensions falling outside the above range make it difficult to provide a inflatable cushion 14 that is large enough to provide adequate support and small enough to be folded and stored within the inner compartment 16. It is also critical to the operation of the kit 10 that the inflatable cushion 14 is made from a highly flexible material and thin material such as medium grade PVC plastic so that when deflated the inflatable cushion 14 can be easily folded and stored within the inner compartment 16.

The foregoing description is intended primarily for purposes of illustration. This invention may be embodied in other forms or carried out in other ways without departing from the spirit or scope of the invention. Modifications and variations still falling within the spirit or the scope of the invention will be readily apparent to those of skill in the art.

What is claimed is:

1. A portable lumbar support kit for use by a person wearing a belt to support the lumbar region of the person's back, the kit comprising:
(a) a pouch having an inner compartment and a clip member engageable with the belt of the person; and
(b) an inflatable cushion folded and stored within said pouch, said cushion may be unfolded and inflated by the person to support the lumbar region of the person's back.

2. The lumbar support kit of claim 1, wherein said inflatable cushion comprises first and second air valves adapted to inflate and deflate said inflatable cushion.

3. The lumbar support kit of claim 2, wherein said first air valve is adapted to allow the person to inflate said inflatable cushion.

4. The lumbar support of claim 3, wherein said second valve is adapted to allow the person to quickly deflate said inflatable cushion.

5. The lumbar support of claim 4, wherein said inflatable cushion may be folded when deflated and stored in said inner compartment of said pouch.

6. The lumbar support kit 5, wherein said pouch further comprises a cover engageable with said inner compartment from an open position where said inflatable cushion may be removed and a closed position where said inflatable cushion is retained within said inner compartment.

7. The lumbar support kit of claim 6, wherein said inner compartment comprises a front wall and a rear wall.

8. The lumbar support kit of claim 7, wherein said cover extends from said rear wall of said inner compartment and is removably engaged with said front wall of said inner compartment by hook and loop fasteners.

9. The lumbar support kit of claim 8, wherein said inflatable cushion is in the form of an elongated cylinder.

10. The lumbar support kit of claim 9, wherein said pouch has a height of less than 3.0 inches.
